(19) United States
(12) Patent Application Publication
(10) Pub. No.: US 2018/0003547 A1
(43) Pub. Date: Jan. 4, 2018

(54) APPARATUS AND METHOD FOR MONITORING CHANGES IN USER WEIGHT

(71) Applicant: KONINKLIJKE PHILIPS N.V., EINDHOVEN (NL)

(72) Inventors: Warner Rudolph Theophile Ten Kate, Waalre (NL); Alberto Giovanni Bonomi, Eindhoven (NL); Gabriele Papini, Eindhoven (NL)

(21) Appl. No.: 15/546,683

(22) PCT Filed: Jan. 27, 2016
(86) PCT No.: PCT/EP2016/051609

§ 371 Date: Jul. 27, 2017

(30) Foreign Application Priority Data
Feb. 3, 2015 (EP) 15153635.6

(51) Int. Cl. G01G 19/44; A43B 3/00; A61B 5/11

(52) U.S. Cl. CPC G01G 19/44; A43B 3/0005; A61B 5/1118

(57) ABSTRACT

Apparatus and methods to monitor changes in user weight. The apparatus includes at least one sensor configured to capture superquotidien measurements of the user's weight and an accelerometer to measure movements of the user, and estimates cumulative weight of the user based on the superquotidien measurements, wherein only sudden changes in the user's weight are used in the estimation. The sensor(s) can be located, e.g., within a shoe insole, within a chair, within a floor, etc.

### FIELD OF THE INVENTION

The present invention generally relates to the monitoring of body weight, and more specifically to the monitoring of body weight utilizing sensors that provide frequent weight measurements.

### BACKGROUND OF THE INVENTION

An active lifestyle and careful nutrition contribute to an improved healthy condition of body and mind. One predictor for a healthy condition is a person's relative size based on weight and height, which can be expressed as a person's body mass index (BMI). As such, a person's weight is a strong determinant of personal health given the strong link between over- and under-weight and disease. In addition, body weight is also a concern of people when perceiving unsatisfactory body shape and beauty. Methods to modify body weight for aesthetical or medical reasons often involve the creation of a negative or positive energy balance where energy expenditure exceeds caloric intake or vice versa.

One popular device for measuring weight is a body weight scale. This device provides a measurement of a user's weight and hence allows measurement of changes therein. However, these measurements are user-initiated and typically occur intermittently over long periods. These infrequent measurements mask the daily changes in weight attributable to events other than food (calorie) intake. Such events include, for example, the weight loss related to fluid loss linked to metabolic and regulatory processes involving energy expenditure. These prior art techniques effectively measure only the day's “net change” in body weight.

Relating these net changes in body weight to calorie intake usually requires the user to input in a kind of electronic diary the food he has been consuming. The diary entries are then converted into an estimate of the calorie intake. One problem with this approach is that it requires active effort from the user. A user may find this process cumbersome or may simply forget to track food he has been consuming.

WO 2010/096691 A2 discloses a footwear system for monitoring weight, posture allocation, physical activity classification, and energy expenditure calculation including an accelerometer configured to obtain acceleration data indicative of movement of a user's foot or leg. The footwear system may also include a pressure sensing device mounted in an insole and configured to obtain pressure data indicative of pressure applied by a user's foot to the insole, as well as a transmitter communicatively coupled to both the accelerometer and the pressure sensing device and configured to transmit the acceleration and pressure data to a first processing device configured process the acceleration data and the pressure data to distinguish a first posture from a second posture different from the first posture and process the acceleration data and the pressure data to distinguish a first movement-based activity from a second movement-based activity different from the first movement-based activity. The footwear system may also include a second processing device communicatively coupled to the first processing device and configured to derive a second energy expenditure value.

US 2006/143645 A1 discloses methods and systems for determining speed or distance traveled of moving persons by utilizing sensors in shoes and for determining and reporting weight of a person wearing a shoe via a sensor with the shoe. Further, shoe based systems employing sensors (e.g., accelerometers) are disclosed to determine and report (e.g., via a watch) speed and/or distance traveled.

### SUMMARY OF THE INVENTION

It is an object of the present invention to provide an apparatus, a method and computer program product for monitoring changes in weight of the user, such as weight changes due to food and drink consumption/intake.

According to the first aspect of the invention this object is achieved by an apparatus for monitoring changes in user weight, the apparatus comprising: a receiver component configured to receive superquotidien weight measurement data of a user; a weight estimation component configured to estimate cumulative weight of the user based on the superquotidien weight measurement data, wherein only sudden changes in the user's weight are used in the estimation; and a comparison component configured to compare the estimated cumulative weight of the user with a prior estimated cumulative weight of the user and detect any change in the cumulative weight of the user.

According to the second aspect of the present invention, a method for monitoring e changes in weight of the user is disclosed. This method includes receiving superquotidien data from a sensor, estimating cumulative weight of the user based on the superquotidien weight measurement data (also called sensor data), wherein only sudden changes in the user's weight are used in the estimation, and comparing the estimated cumulative weight of the user with a prior estimated cumulative weight of the user and detecting any change in the cumulative weight of the user. The more frequent weight measurements have the advantage of tracking individual events affecting weight throughout the day.

In various embodiments of the invention, the term “superquotidien” means “more than once a day; typically at least once an hour; potentially more often or even continuous; potentially less often than once an hour.”

The apparatus and method as described above has numerous advantages. Firstly, the person/user need not use a conventional scale every time he needs to check his weight. Given that the weight measurements are periodic in nature, it benefits the user to understand his overall weight/net weight (also called “cumulative weight” or “cumulative sum of the user's weight changes” herein) efficiently. Additionally, the weight or the net weight thus calculated is based on filtering various events that may give false sense of weight addition, such a wearing a coat, holding a heavy object, etc. Hence, the weight calculation is thus more accurate. In general, the apparatus and method, automatically detect changes in body weight for subsequent use in a variety of applications, such as determining calorie intake. The automatically collected body weight information can be converted to a record of user activity and effectively guide a user towards management of body weight.

Additionally, the measurements that are stored in the memory device can include all weight measurements or only those measurements that are greater in magnitude than a predetermined minimum weight change over a particular (predetermined) time period of interest, which is also referred to herein as a “sudden” change of weight. In particular, the weight estimation component may be used to identify sudden changes in weight.

In various embodiments, the weight estimation component estimates the cumulative weight by identifying sudden weight increases in the superquotidien weight measurement data and summing those sudden weight increases to generate the cumulative weight. In some embodiments, the weight estimation component additionally identifies sudden weight decreases in the superquotidien weight measurement data and additionally sums the sudden weight decreases to generate the cumulative weight. In other words, the cumulative weight may include a sum of sudden weight changes, including both increases and decreases.

In a preferred embodiment, the user's weight is measured continuously, for example by using pressure insoles. Those measurements are contaminated with noise as well as fluctuations by the user's movements, which will put a lower boundary on the resolution of the measurements. Filters and other techniques can be applied to reduce the noise and provide a best estimate of the actual weight. For the sake of explanation it is assumed the measurements are noise-free. A sudden change then is the change in weight because, for example, the user is picking up a glass of water, or is putting on a coat. A sudden change also happens when the user visits the toilet, or puts off his coat again, or places the cup of coffee back on the table. The first examples cause (sudden) weight increments, the latter decrements. Regular metabolism also causes weight to lower, through sweating and respiration. This change is an example of a non-sudden change. Hence, sudden changes are those that happen instantaneously, like lifting a cup, or that happen in a couple of minutes, like eating some food, or visiting the toilet.

The change is sustained: the weight before the change is considered constant, as it is after the change, where “before” and “after” can be a couple of minutes apart. Since in practice, measurements are not continuous, and might even happen irregularly, also because some measurement data might be too noisy/unreliable, “sudden” shall be understood as covering the used sample interval, and the “immediate” changes (e.g. lifting a cup) and changes in a “couple of minutes” (e.g. eating food, toilet) are both happening within the duration of the sample interval and shall thus be understood as sudden changes. Sudden changes may also be understood as changes having some form of abruptness, i.e. changes that do not happen gradually: the time series of weight measurements has a breakpoint, and are “constant” before and “constant” after; mathematically a (higher order) derivative is discontinuous.

Weight increment by consumption/calorie intake may be monitored according to the present invention. In an embodiment this is done by observing the daily (or other periodically) cumulation of sudden changes in user weight as they happen over the day (or other period). Thus, the weight change estimation component may be configured to estimate the daily/periodically weight change of the user based on the cumulation of changes in the superquotidien weight measurement data. Further, the comparison component may be configured to compare the estimated periodically weight change of the user with a prior estimated periodically weight change of the user and detect any change in periodically weight change of the user.

In another embodiment the apparatus further comprises a weight tracking component used to track the changes in the user's cumulative weight based on the superquotidien weight measurement data. The tracking uses a trend (i.e. a series of previous measurements). Comparison includes testing on changes in trend. A single deviation is not immediately a reason to alert. If comparison is with previous day and behavior changes, there is a change only once, the next days are comparable to their previous days. A trend line obviously makes a step, not a single outlier.

To further elaborate, embodiments of the present invention generally relate to the monitoring of body weight, and more specifically to the monitoring of body weight utilizing sensors that provide frequent weight measurements. The more frequent weight measurements have the advantage of tracking (i.e. monitoring or computing) individual events affecting weight throughout the day. The weight measurement data can be all weight measurements or only those measurements that are a “sudden” change of weight (as defined above). This has the advantage of filtering gradual or noisy changes in the weight measurement.

Embodiments of the inventive apparatus and method (described below) utilize a sensor to acquire frequent (i.e., considerably more often than once a day) measurements of user weight. This allows the detection of small changes in weight, and in particular sudden changes, as they occur over the day. The cumulative (i.e. summed) history of these weight changes allows, e.g., the estimation of the user's calorie intake over the day.

This is an improvement over the prior art in that net weight change computed from daily weight measurements includes losses due to energy expenditure and therefore could not provide an estimate of the user's calorie intake for the day. Moreover, the net weight change computed from daily weight measurements is of the same order as (or even less than) the accuracy of the measurement itself.

In another embodiment of the present invention, the apparatus for monitoring changes in a user's cumulative weight as set out above is disclosed along with the receiver component being further configured to receive secondary user data. This secondary user data can include movement data, temperature data, water loss data, resting metabolic rate data, filtered measurements of the user's weight, and the time that has lapsed since the last measurement of the user's weight. This secondary user data has the advantage of enabling more accurate weight estimates that account for factors that affect user weight.

In another embodiment of the invention, a calorie estimation component can be included to estimate the user's calorie intake based on the detected change in cumulative weight of the user.

In another embodiment of the invention, the weight estimation component can also be configured to estimate the cumulative weight of the user based on the superquotidien weight measurement data and the secondary user data. Moreover, the weight estimation component can be configured to estimate the cumulative weight of the user based on the superquotidien weight measurement data and at least one of a prior estimated cumulative weight of the user, movement data of the user, environmental temperature data, and environmental humidity data. This has the advantage of enabling more accurate weight estimates that account for factors that affect user weight.

In another embodiment of the present invention, the apparatus for monitoring changes in a user's cumulative weight as set out above is disclosed along with a correction component configured to estimate the cumulative weight of the user based on the superquotidien weight measurement data and the secondary user data in the event there is insufficient weight measurement data. This has the advantage of enabling accurate weight change estimates in the absence of a direct measurement of weight.

In another embodiment of the present invention, the apparatus for monitoring changes in a user's cumulative weight as set out above is disclosed along a user interface configured to receive input from the user. In yet another embodiment of the present invention, the apparatus for monitoring changes in a user's cumulative weight as set out above is disclosed along a sensor located within a shoe insole, a chair, a floor, in undergarments, or in proximity to a doorsill. In still another embodiment of the present invention, the apparatus for monitoring changes in a user's cumulative weight as set out above is disclosed along at least one sensor selected from the group consisting of a pressure sensor, a weight sensor, and a force sensor.

In another embodiment, the apparatus for monitoring changes in a user's cumulative weight as set out above is disclosed along at least one accelerometer configured to measure the movements of the user, wherein the measurements of the at least one accelerometer are used to filter the measurements of the at least one sensor. This has the advantage of permitting the rejection of weight sensor measurements that may be inaccurate due to the wearer's motion or lack thereof.

In another embodiment of the present invention, the method for monitoring changes in a user's cumulative weight as set out above is disclosed along applying a correction factor to the estimated weight of the user. The correction factor can be based on movement data, temperature, water loss, resting metabolic rate, filtered measurements of the user's weight, and/or the time lapsed since the last measurement of the user's weight in the event there is insufficient sensor data. This correction factor has the advantage of enabling more accurate weight estimates that account for factors that affect user weight.

Another aspect of an embodiment of the present invention includes an apparatus to record user data. The embodiment includes at least one sensor configured to measure a user's weight and a memory device configured to store measurements of the user's weight. The sensor is used to capture superquotidien measurements of the user's weight. These measurements are stored in the memory device and then can be used to track the changes in the user's cumulative weight based on the superquotidien measurements of the user's weight.

In one embodiment, the apparatus further includes a correction component configured to apply a correction factor to the measurements of the user's weight, the correction factor being derived from at least one factor selected from the group consisting of movement data, temperature, water loss, resting metabolic rate, filtered measurements of the user's weight, and the time lapsed since the last measurement of the user's weight.

The at least one sensor can be located within a shoe insole, a chair, a floor, in undergarments, in proximity to a doorsill, or other locations in which superquotidien measurements of a user can be taken. Further, the sensor and the memory device can be co-located within the same apparatus or the same location. The sensor can be selected from known sensors that can produce a weight measurement, such as a pressure sensor, a weight sensor, and a force sensor.

In another embodiment of the present invention, the apparatus to record user data above is disclosed along with at least one accelerometer configured to measure the movements of the user. These measurements can be used to filter or otherwise control and modify the measurements of the sensor.

In another aspect, the invention relates to a computer readable medium containing computer-executable instructions for performing a method for monitoring changes in user weight when executed by a processor.

In another aspect, the invention relates to a computer program which comprises program code means for causing a computer to perform the steps of the method for monitoring changes in user weight when said computer program is carried out on a computer.

The embodiments of the method aspect discussed above may be implemented as computer-executable instructions on a computer-readable medium in accord with this aspect of the present invention. The computer-readable medium may be suited to physical transfer, such as a CD-ROM or USB storage device, or it may be embedded within an apparatus, such as a memory contained within a computer or smartphone that is executed by a processor and subsequently interacts with, e.g., a sensor.

These and other features and advantages, which characterize the present non-limiting embodiments, will be apparent from a reading of the following detailed description and a review of the associated drawings. It is to be understood that both the foregoing general description and the following detailed description are explanatory only and are not restrictive of the non-limiting embodiments as claimed.

### BRIEF DESCRIPTION OF DRAWINGS

Non-limiting and non-exhaustive embodiments are described with reference to the following figures in which:

FIG. 1 is an example of micro-weighing in accord with the present invention;

FIG. 2 is a schematic representation of an embodiment of an apparatus to record weight data according to the present invention;

FIG. 3 is a schematic representation of an embodiment of an apparatus to monitor changes in cumulative weight according to the present invention;

FIG. 4 is a graph of weight over time as it changes in response to various metabolic processes; and

FIG. 5 is a schematic representation of an embodiment of a method to monitor changes in cumulative weight according to the present invention.

In the drawings, like reference characters generally refer to corresponding parts throughout the different views. The drawings are not necessarily to scale, emphasis instead being placed on the principles and concepts of operation.

### DETAILED DESCRIPTION OF THE DRAWINGS

Various embodiments are described more fully below with reference to the accompanying drawings, which form a part hereof, and which show specific exemplary embodiments. However, embodiments may be implemented in many different forms and should not be construed as limited to the embodiments set forth herein; rather, these embodiments are provided so that this disclosure will be thorough and complete, and will fully convey the scope of the embodiments to those skilled in the art. Embodiments may be practiced as methods, systems or devices. Accordingly, embodiments may take the form of a hardware implementation, an entirely software implementation or an implementation combining software and hardware aspects. The following detailed description is, therefore, not to be taken in a limiting sense.

Reference in the specification to “one embodiment” or to “an embodiment” means that a particular feature, structure, or characteristic described in connection with the embodiments is included in at least one embodiment of the invention. The appearances of the phrase “in one embodiment” in various places in the specification are not necessarily all referring to the same embodiment.

Some portions of the description that follow are presented in terms of symbolic representations of operations on non-transient signals stored within a computer memory. These descriptions and representations are the means used by those skilled in the data processing arts to most effectively convey the substance of their work to others skilled in the art. Such operations typically require physical manipulations of physical quantities. Usually, though not necessarily, these quantities take the form of electrical, magnetic or optical signals capable of being stored, transferred, combined, compared and otherwise manipulated. It is convenient at times, principally for reasons of common usage, to refer to these signals as bits, values, elements, symbols, characters, terms, numbers, or the like. Furthermore, it is also convenient at times, to refer to certain arrangements of steps requiring physical manipulations of physical quantities as modules or code devices, without loss of generality.

However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices.

Certain aspects of the present invention include process steps and instructions that could be embodied in software, firmware or hardware, and when embodied in software, could be downloaded to reside on and be operated from different platforms used by a variety of operating systems.

The present invention also relates to an apparatus for performing the operations herein. This apparatus may be specially constructed for the required purposes, or it may comprise a general purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability. The present invention may also take the form of the computer program itself stored in the computer readable storage medium prior to or after its download and/or execution.

The processes and displays presented herein are not inherently related to any particular computer or other apparatus. Various general purpose systems may also be used with programs in accordance with the teachings herein, or it may prove convenient to construct more specialized apparatus to perform the required method steps. The required structure for a variety of these systems will appear from the description below. In addition, the present invention is not described with reference to any particular programming language. It will be appreciated that a variety of programming languages may be used to implement the teachings of the present invention as described herein, and any references below to specific languages are provided for disclosure of enablement and best mode of the present invention.

In addition, the language used in the specification has been principally selected for readability and instructional purposes, and may not have been selected to delineate or circumscribe the inventive subject matter. Accordingly, the disclosure of the present invention is intended to be illustrative, but not limiting, of the scope of the invention, which is set forth in the claims.

### Superquotidien User Weight Measurement

Embodiments of the present invention generally relate to the monitoring of user weight, and more specifically, to the monitoring of user weight utilizing sensors that provide frequent (i.e., considerably more often than once a day) measurements of user weight.

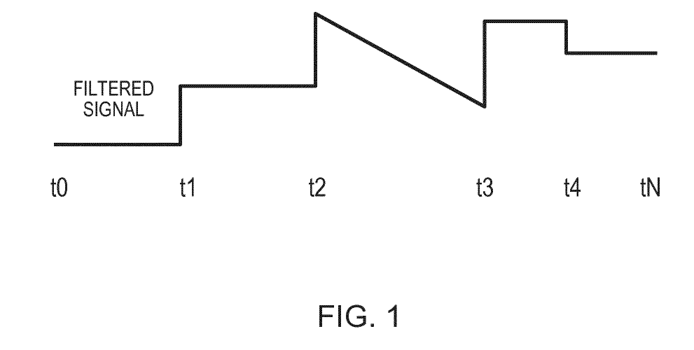

FIG. 1 illustrates how body weight can vary over the course of the day as a user engages in various daily living activities. At time t1 a sharp increase in weight is detected. This increase is likely due to an intake of food, but it could also be due to a change of clothing (e.g., dressing, putting on a coat). Various embodiments of the present invention distinguish between these kinds of activities utilizing time of day or other secondary information. At time t2 another sharp increase in weight is detected, which again indicates a food intake event, change of clothing, etc. These sharp increases would be tracked for the calculation of the user's cumulative weight change (i.e. the change of the user's cumulative weight) for the monitored period.

Between t2 and t3 there is a gradual decrease in weight. FIG. 1 depicts a straight line; the gradual decrease could also be curved (e.g., exponential). The gradual decrease is caused by the user's energy expenditure, by their resting metabolism, by explicit exercise, by sweating, etc. This gradual decrease can be discarded for purposes of the calculation of the user's cumulative weight change, as it reflects an autonomic process over which the user exerts little control, i.e. it does not represent a “sudden” change in the user's weight as used in the context of the present invention.

This is one reason embodiments of the present invention are better able to estimate a user's calorie intake and make appropriate recommendations. A prior art system measuring weight once a day would include the loss by energy expenditure in its measurement of net weight change and therefore suggest a lower calorie intake. Moreover, the net weight change value including gradual weight loss could be below the stated variance for the prior art weight sensor.

At time t3 another sharp increase in weight is detected, which again indicates a food intake event, a change of clothes, etc. At time t4 a sharp decrease in weight is detected. This decrease can come from a change in clothing (e.g., undressing, removing a coat), toileting, etc. These sharp decreases are subtracted from the day's cumulative sum of the user's weight changes. In particular, pairs of increases and decreases in weight for the same approximate amount can be offset against each other as they suggest a change in clothing (e.g., putting a coat on and later taking the coat off). The assumption is that weight increment e.g. by dressing is balanced by weight decrement by undressing and that the residue over the day is small. Also, weight loss at the toilet is assumed to improve the actual weight gain of food intake, i.e. yielding the “effective food intake”. Some embodiments may only consider weight increases in tracking the user's cumulative weight change for the monitored period.

The cumulative sum of the user's weight change (i.e. the “cumulative weight” of the user obtained from the sudden changes of the user's weight, i.e. the sudden changes at t1, t2, t3 and t4 in the example shown in FIG. 1) over the monitored period is useful for a variety of applications. For example, the cumulative sum can be used to estimate the calorie intake for the day based on average intake for that particular user or for that particular type of user (e.g., having the same gender, body type, activity level, types of activities, BMI, eating habits (consumption of fried foods, fruit, etc.).

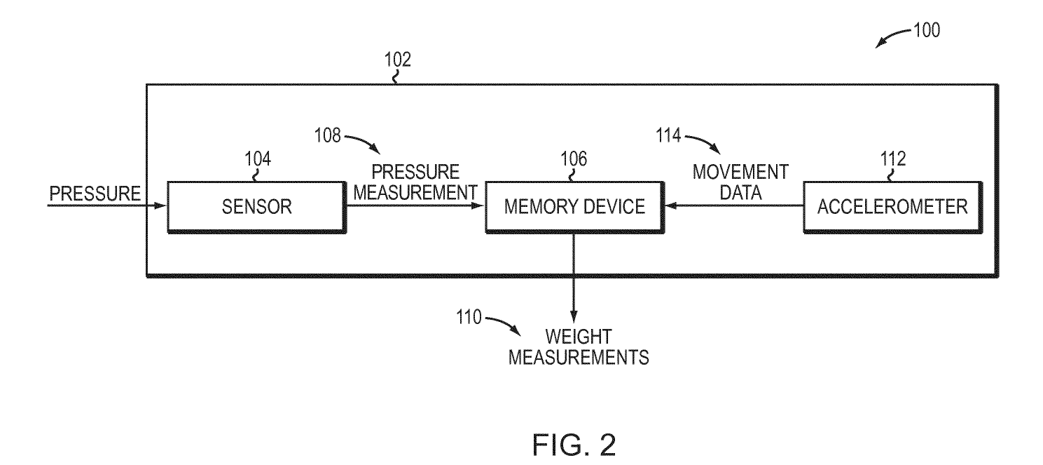

FIG. 2 presents a schematic representation 100 of an apparatus to record user data 102 in accordance with one embodiment of the invention. User data recording apparatus 102 has at least one sensor 104 configured to measure and capture superquotidien (i.e., considerably more often than once a day) measurements of weight 108 exerted thereon and a memory device 106 configured to store measurements of the weight 108. The measurements of weight 108 can be used to calculate and track the changes in the user's cumulative weight 110.

The sensor 104 may be positioned in a variety of locations, such as within a shoe insole, a chair, a floor, in undergarments, in proximity to a doorsill, etc. Suitable sensors 104 include pressure sensors, weight sensors, force sensors, etc.

In this embodiment a single sensor is illustrated. However, one skilled in the art will recognize that a plurality of sensors may be used and, as such, disclosure of a single sensor is illustrative and is not meant to be limiting. Further, one skilled in the art will recognize that various types of sensors can be utilized to capture weight measurements in accord with the present invention, including but not limited to pressure sensors, weight sensors, and force sensors.

With the introduction of a plurality of sensors, each sensor can be used to verify the weight measurement provided by the other sensors, discarding measurements that cannot be confirmed or otherwise appear to be outliers.

Cumulative weight measurements (estimates) 110 can be affected by various secondary factors or events 120. Such factors and events include user movement, water loss, temperature, resting metabolic rate, filtered measurements of the user's weight, and the time lapsed since the last measurement of the user's weight. In the embodiment shown, for example, an accelerometer 112 is used to provide movement data 114 that indicates whether the measurements 108 from sensor 104 are suitable for use in the calculation of cumulative weight measurements 110.

For example, with an accelerometer the direction of gravity can be estimated, and only when that direction is vertical the corresponding weight samples 108 are used. Similarly, or in addition, weight samples 108 may be used if the accelerometer 112 indicates that the user's activity level is low, i.e., when there is little variance in the acceleration signal. In still other embodiments, the level of variance in the acceleration signal may be used to correct the measured weight 108. In yet another embodiment, the magnitude of the acceleration signal can be monitored and when it is close to 1 g, indicating the user is “at rest,” then the weight samples 108 may be utilized. Note, however, that during walking when the user's foot is on the ground a constant 1 g will be measured, while the weight samples 108 include an additional ground reaction force from the user's walking.

As mentioned above, the illustration of a single sensor is not limiting and an accelerometer is one type of sensor. Therefore, one skilled in the art will recognize that a plurality of accelerometers along with additional types of measurement devices and sensors can be utilized to capture secondary user data.

### Filtered Weight Measurements

When the user's weight is measured at a high sampling rate, additional filtering is possible to arrive at a better estimate of the user's “true” weight. For example, the use of a linear low-pass filter or non-linear filters such as median and mode filters can be utilized.

A median filter is known to stop pulses and oscillations, while passing through constants, trends and edges. Unlike linear filters, median filters don't exhibit transients when the stationarity changes in the measured weight signal. Other filter forms of the median filter include the recursive median filter. This filter has the property that it tends to maintain its previous estimates of weight. In this way, a fluctuation in the weight measurement signal does not immediately appear in the estimate, while a change (i.e., an edge), e.g. due to food intake or toileting, is still incorporated.

Other filter structures include a mode filter and a so-called “sub-median” filter. Variations of a median filter, such as a weighted median filter, can also be used. Where the median filter takes the median value of all sample values in the current window, a mode filter takes the value of the most frequently occurring sample value in the current window.

The sub-median filter is a combination of non-linear and low-pass filters. In addition, this non-linear filter operates at a subsampled rate, has good estimation properties for the DC component, and is responsive to fast changes in the DC signal. In general, the output of a low-pass filter will converge on the DC value, but with a somewhat slow response time. A median filter eliminates significant deviations of short duration, like spikes, from the signal while it follows edges and preserves detail.

### Cumulative Weight Change Tracking

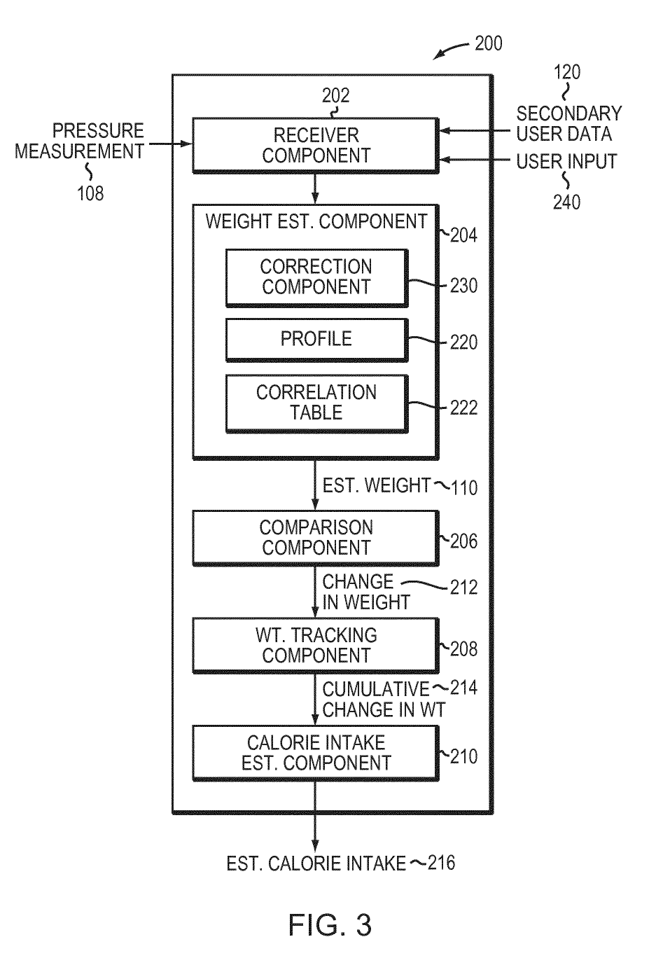

Referring to FIG. 3, a schematic representation of an apparatus for monitoring changes in a user's cumulative weight 200 in accordance with one embodiment of the invention is illustrated. Cumulative monitoring apparatus 200 has a receiver component 202, a weight estimation component 204, a comparison component 206, a weight tracking component 208 and a calorie estimation component 210.The apparatus 200 can take the form of, e.g., a processor executing a stored program to provide the functionality of these various components or it can be a collection of discrete hardware elements, with one or more of each of those elements providing the functionality of these various components.

Receiver component 202 is configured to receive superquotidien measurements of weight 108 and secondary user data 120 such as those discussed above in connection with FIG. 2. Receiver component 202 may take the form of, e.g., an application specific integrated circuit (ASIC), a general purpose computing element with one or more input lines in communication with a sensor, or a processor programmed to receive various data values.

The weight estimation component 204 is configured to estimate the cumulative weight of the user 110 based on superquotidien measurements of weight 108 and secondary user data 120, along with profile information 220 and calibration tables 222. In this embodiment, profile information 220 can include information such as the user's gender, body mass index and eating habits, e.g., preferences for fried foods, fruit, etc. Calibration tables 222 can be parameterized on factors like weight, height, gender and time of day. Weight estimation component 204 may take the form of, e.g., an application specific integrated circuit (ASIC) or a general purpose computing element programmed to receive various data values and provide an estimated cumulative weight (i.e. a cumulative sum of the user's weight changes).

Comparison component 206 is configured to compare the estimated cumulative weight of the user 110 with a prior estimated cumulative weight of the user and detect any change in cumulative weight of the user 212. The weight tracking component 208 is used to track the change in the user's cumulative weight 214. A calorie estimation component 210 is used to estimate the user's calorie intake 216 based on the tracked change in cumulative weight of the user 214. Each of these components 206—may take the form of, e.g., an application specific integrated circuit (ASIC) or a general purpose computing element programmed to provide the described functionality.

As mentioned above, secondary user data 120 may take various forms, including movement data, temperature data, water loss data, resting metabolic rate data, filtered measurements of the user's weight, the time that has lapsed since the last measurement of the user's weight, environmental temperature data, environmental humidity data, and so forth.

In another embodiment of the present invention, the apparatus for monitoring changes in a user's cumulative weight as set out above is disclosed with a user interface 240 configured to receive input from a user. Utilizing the user interface 240, the user can explicitly specify that he has consumed food or drink and of what kind. This information can be used to train the system, both in terms of identifying relevant weight changes (i.e., the time, size and speed of the change) as well as labelling the associated calorie intake (given the type of comestible consumed). Additionally, when a change in weight has been detected, the inventive apparatus can request, via the user interface 240, the user to confirm the intake as well as to supplemental information about the type of food or drink consumed. Additional embodiments of the apparatus of the present invention have the capability to detect dehydration of and the resting metabolic rate of the user.

Estimating User Weight with Stale or Sparse Measurements

In this embodiment, weight estimation component 204 includes a correction component 230 that is configured to estimate the weight of the user in the event there is insufficient weight measurement data 108. This estimation can be based on, e.g., an interpolation of previously received weight data adjusted for historical records of weight change correlated with, e.g., time of day, etc. Since the apparatus of the present invention measures and records sudden weight changes, in one embodiment the interpolation consists of holding the last weight measurement, and doing so yields the net weight change with respect to the current sample.

As the interpolation is an estimate, an error term remains. As many of these micro-weights may be on the order of tens of grams (or at most a few hundred grams), the amount of permissible variance in the interpolated estimate is small. The problem of accurately estimating the cumulative weight change in the absence of sufficient weight measurements remains. A refinement to the interpolation to address this problem accounts for: (1) the weight loss that normally results from CO₂ exhalation associated with the metabolic processes, and (2) water loss through respiration and skin evaporation.

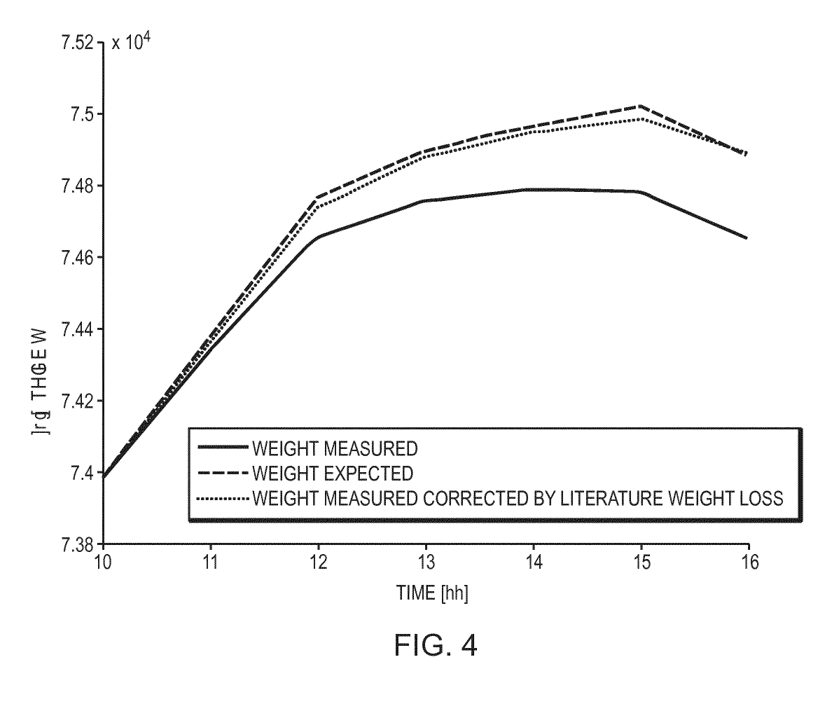

FIG. 4 depicts an example of how body weight changes over the course of six hours in response to these two factors. The “weight measured” line is the actual weight measurement of the user with time. The “weight expected” line over time is the cumulative sum of the user's initial body weight plus or minus the real weight value of elements taken into the body (e.g. food) or expelled from the body (e.g., bowel movements or other bodily functions).

The difference shown between the “weight measured” and the “weight expected” is due to the aforementioned biological factors and, as expected, it grows greater with time. In this example, the weight loss is known to be about 40 grams/hour in an average person, mostly due to water loss. If last weight measurement is two hours old, and there is no intervening measurement data, the current estimate of the user's cumulative weight should be the weight as measured two hours ago minus 80 grams.

Estimates of user's cumulative weight in the absence of recent weight measurements can be further improved with additional information. For example, the user's resting metabolic rate can be directly or indirectly measured and used to adjust the estimate of the user's cumulative weight in lieu of the 40 grams/hour figure, which is an average figure.

In another embodiment, a record of activity data from an accelerometer can be used to adjust the estimated RMR for the user with the energy expended as measured by the accelerometer, and the adjusted RMR can be used to adjust the estimate of the user's cumulative weight in addition to or in lieu of adjustments for water loss, CO₂ exhalation, etc.

In some embodiments, available (though out-of-date) weight samples may be smoothed or interpolated using signal processing techniques to estimate the user's likely current weight. Depending on their nature, non-linear filtering techniques can be superior to linear ones for these purposes.

Interpolated weight values can be used to test for possible sudden changes in the missing data sequence. For example, a user's cumulative weight would normally decline with time due to water loss, etc., as discussed above. Accordingly, a subsequent weight measurement (w₂) would normally be less than a preceding weight measurement (w₁) by an amount that would normally be proportional to the difference in time between the two weight measurements, i.e., k*(t₂−tᵢ). If the subsequent measurement is greater than the weight measurement expected for that later point in time, then an embodiment of the present invention can infer that a sudden weight change may have occurred in the interim. Moreover, the difference between the subsequent measurement and the expected value for that measurement can be used to infer the magnitude of the change. The computed magnitude of the change can also be compared to be a list of stored changes to identify the kind and/or nature of the change.

The accumulated weight change can also be combined with the aforementioned weight estimate corrected for metabolic rate and other factors, as discussed above. One way to combine these two estimates can involve weighting them according to their respective variances, so that the estimate with the lowest variance (i.e., the estimate most likely to be correct) receives the most weight.

Tracking Cumulative User Weight with Superquotidien Weight Measurements

As presented above, embodiments of an apparatus to record user weight data and an apparatus for monitoring changes in a user's cumulative weight in accordance with the present invention have been illustrated as being separate for simplicity of discussion. One skilled in the art will recognize that all or parts of these apparatuses can themselves contained in a single embodiment.

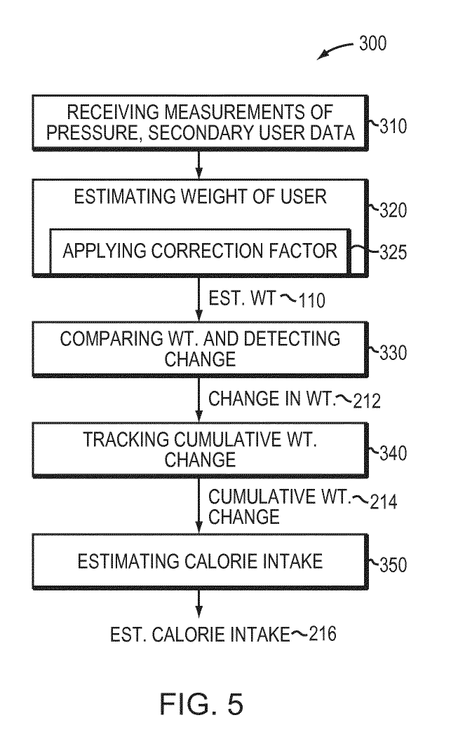

Referring to FIG. 5, a schematic representation 300 of a method for monitoring changes in a user's cumulative weight in accordance with one embodiment of the invention is illustrated. In step 310, a receiving component 202 receives superquotidien measurements of user weight 108 and secondary user data 120.

In step 320, a weight estimation component 204 estimates the cumulative weight of the user 110 based on the superquotidien measurements of weight 108 and secondary user data 120. Additionally, a correction factor may be applied (Step 325) to the estimated cumulative weight 110 to improve the accuracy of the estimate. The correction factor can be based on movement data, temperature, water loss, resting metabolic rate, filtered measurements of the user's weight, and/or the time lapsed since the last measurement of the user's weight in the event there is insufficient sensor data.

In step 330, a comparison component 206 compares the estimated cumulative weight of the user 110 with a prior estimated cumulative weight of the user to identify any change in the cumulative weight 212. In step 340, a weight tracking component 208 tracks the changes in the user's cumulative weight 212. In step 350, an optional calorie estimation component 210 estimates the user's calorie intake 216 based on the tracked change in weight of the user 214.

Energy expenditure is conventionally estimated from metrics like amount of energy in an accelerometer signal (e.g. of an accelerometer worn by the user). Given weight change over a day and given energy expenditure, an estimate of the calorie intake can be derived, which would be an implicit measurement. The sum of sudden weight changes, as used according to the present invention, provides a (more) explicit estimate since they directly relate to (measurements at) intake moments.

A user can be informed by two independent numbers (one is not derived/implied from the other): the calorie intake and energy expenditure. Consequently, the user can decide to improve his weight loss program by being more active or by further reducing his consumption, for instance.

The measurements inform about weight increment, not immediately about calorie intake. A translation can be made between these two. To do so, the distribution of types of food, i.e. the average amount of calorie per kg has to be known for that user. This conversion ratio can be taken from general population numbers or be made tailored for that user. It may also suffice to inform the user about his cumulative weight change, without transforming into a calorie number. The user may recall what type of food has been eaten and adjust his further consumption.

Embodiments of the present disclosure, for example, are described above with reference to block diagrams and/or operational illustrations of methods, systems, and computer program products according to embodiments of the present disclosure. The functions/acts noted in the blocks may occur out of the order as shown in any flowchart. For example, two blocks shown in succession may in fact be executed substantially concurrent or the blocks may sometimes be executed in the reverse order, depending upon the functionality/acts involved. Additionally, not all of the blocks shown in any flowchart need to be performed and/or executed. For example, if a given flowchart has five blocks containing functions/acts, it may be the case that only three of the five blocks are performed and/or executed. In this example, any of the three of the five blocks may be performed and/or executed.

The description and illustration of one or more embodiments provided in this application are not intended to limit or restrict the scope of the present disclosure as claimed in any way. The embodiments, examples, and details provided in this application are considered sufficient to convey possession and enable others to make and use the best mode of the claimed embodiments. The claimed embodiments should not be construed as being limited to any embodiment, example, or detail provided in this application. Regardless of whether shown and described in combination or separately, the various features (both structural and methodological) are intended to be selectively included or omitted to produce an embodiment with a particular set of features. Having been provided with the description and illustration of the present application, one skilled in the art may envision variations, modifications, and alternate embodiments falling within the spirit of the broader aspects of the general inventive concept embodied in this application that do not depart from the broader scope of the claimed embodiments.

1. An apparatus for monitoring changes in user weight, the apparatus comprising:
a receiver component configured to receive superquotidien weight measurement data of a user representing weight measurement data measured more than once a day;
a weight estimation component configured to estimate cumulative weight of the user based on the superquotidien weight measurement data, wherein only sudden changes in the user's weight are used in the estimation; and
a comparison component configured to compare the estimated cumulative weight of the user with a prior estimated cumulative weight of the user and detect any change in the cumulative weight of the user.

2. The apparatus of claim 1 wherein the receiver component is further configured to receive secondary user data, wherein the secondary user data comprises movement data of a user, temperature data of the user, water loss data of the user, resting metabolic rate data of the user, filtered measurement of the user's weight, and the time lapsed since the last measurement of the user's weight.

3. (canceled)

4. The apparatus of claim 1 further comprising a calorie estimation component configured to estimate the user's calorie intake based on the detected change in cumulative weight of the user.

5. The apparatus of claim 2, wherein the weight estimation component is further configured to estimate the cumulative weight of the user based on the superquotidien weight measurement data and the secondary user data.

6. The apparatus of claim 2, wherein the weight estimation component is configured to estimate the cumulative weight of the user based on the superquotidien weight measurement data and at least one of a prior estimated cumulative weight of the user, movement data of the user, environmental temperature data, and environmental humidity data.

7. The apparatus of claim 2 further comprising a correction component configured to estimate the cumulative weight of the user based on the superquotidien weight measurement data and the secondary user data in the event there is insufficient weight measurement data.

8. The apparatus of claim 1 further comprising a user interface configured to receive input from the user and/or a weight tracking component used to track the changes in the user's cumulative weight based on the superquotidien weight measurement data.

9. The apparatus of claim 1 further comprising at least one sensor located within a shoe insole, a chair, a floor, in undergarments, or in proximity to a doorsill.

10. The apparatus of claim 9, wherein the at least one sensor is selected from the group consisting of a pressure sensor, a weight sensor, and a force sensor.

11. The apparatus of claim 10 further comprising at least one accelerometer configured to measure the movements of the user, wherein the measurements of the at least one accelerometer are used to filter the measurements of the at least one sensor.

12. A method for monitoring changes in user weight, the method comprising:
receiving superquotidien weight measurement data from a sensor representing weight measurement data measured more than once a day;
estimating cumulative weight of the user based on the superquotidien weight measurement data, wherein only sudden changes in the user's weight are used in the estimation; and
comparing the estimated cumulative weight of the user with a prior estimated cumulative weight of the user and detecting any change in the cumulative weight of the user.

13. The method of claim 12 further comprising applying a correction factor to the estimated cumulative weight of the user based on at least one factor selected from the group consisting of movement data, temperature, water loss, resting metabolic rate, filtered measurements of the user's weight, and the time lapsed since the last measurement of the user's cumulative weight in the event there is insufficient weight measurement data.

14. A non-transitory computer readable medium containing computer-executable instructions for performing a method for monitoring changes in user weight as defined in claim 12, when executed by a processor.

15. A computer program which comprises program code means for causing a computer to perform the steps of the method for monitoring changes in user weight as defined in claim 12 when said computer program is carried out on a computer.
